(19) United States
(12) Patent Application Publication
(10) Pub. No.: US 2025/0367183 A1
(43) Pub. Date: Dec. 4, 2025

(54) SUBSTANCE FOR PROMOTING REGENERATION AND REPAIR OF ORGANS OF MAMMALS AND USE THEREOF

(71) Applicants: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(72) Inventors: Wei LI, Beijing (CN); Qi ZHOU, Beijing (CN); Zheng-Quan HE, Beijing (CN); Zongbao LU, Beijing (CN); Shuai WANG, Beijing (CN); Xin WANG, Beijing (CN); Xuewei YUAN, Beijing (CN); Liu WANG, Beijing (CN)

(73) Assignees: INSTITUTE OF ZOOLOGY, CHINESE ACADEMY OF SCIENCES, Beijing (CN); BEIJING INSTITUTE FOR STEM CELL AND REGENERATIVE MEDICINE, Beijing (CN)

(21) Appl. No.: 18/875,382

(22) PCT Filed: Jun. 16, 2023
(86) PCT No.: PCT/CN2023/100868

§ 371 Date: Dec. 16, 2024

(30) Foreign Application Priority Data
Jun. 16, 2022 (CN) 202210680476.9

(51) Int. Cl. A61K 31/45; A61K 31/203; A61K 31/381; A61K 31/40; A61K 31/423; A61K 31/5377; A61P 17/02

(52) U.S. Cl. CPC A61K 31/45; A61K 31/203; A61K 31/381; A61K 31/40; A61K 31/423; A61K 31/5377; A61P 17/02

(57) ABSTRACT

Provided is use of a substance capable of up-regulating ISG gene expression in preparing a drug or reagent for promoting the regeneration and repair capacity of tissues or complex structures or organs of mammals. The substance for up-regulating ISG gene expression is selected from one or two or more of an MAPK inhibitor, a retinoic acid receptor-related orphan receptor inhibitor, a protein synthesis inhibitor, and an interferon (IFNγ, β, λ) or alarmin (S100A8/A9) protein. The regeneration and repair refer to promoting the regeneration of tissues or complex structures or organs after tissue or organ resection or damage.

### FIELD OF THE INVENTION

The present invention relates to the field of biotechnology, and in particular to a method for promoting regeneration and repair of a tissue or complex structure or organ of mammal, and use thereof.

### BACKGROUND OF THE INVENTION

Regeneration refers to the repair process in which a structure with the same morphology and function as the lost part grows on the basis of the remaining part after the whole body or an organ of an organism is injured and partially lost. Failure of regeneration can lead to loss of tissue or organ functionality, ultimately causing various diseases or even death. Different species in nature have different regeneration capacities, which can be divided into: 1. regeneration at the individual level, such as lower plants can regenerate a plant from a single cell, some higher plants can use roots, stems, leaves and other tissues to regenerate new plants, lower organisms such as turbellarians can use any part of the body to regenerate a complete individual; 2. regeneration after excision, tailed amphibians such as anacondas, geckos and some fish can regenerate severed tails, limbs and fins; 3. regeneration at the tissue level, such as regeneration achieved through hepatocyte proliferation after liver resection, and regeneration of skin tissue; 4. regeneration at the cell level, such as the regrowth of broken neuronal axons. However, compared with plants and lower animals, mammals, including humans, have greatly lost the capacity to regenerate and have almost no capacity to regenerate after excision. Regeneration is limited to the fetal period and specific tissues or organs, such as the liver and skin, at specific periods. The general trend is: as the evolutionary level increases, the species' regenerative capacities become weaker, or even lost.

When mammals, including humans, face various damages, scar formation occurs as a repair method, which can directly cause tissue and organ dysfunction, such as loss of movement due to amputation, or fibrosis-related diseases, such as cardiovascular disease, degenerative nervous system disease, lung disease, liver disease, diabetes, skin disease, etc. Worldwide, scar formation caused by damage to tissues and organs is the main cause of disability and death from many diseases. Therefore, improving the in situ regeneration and repair capacities of mammals is an important means to understand the essence of life and treat a series of related diseases.

In mammals, severe and chronic damages are usually repaired by scar formation rather than tissue regeneration, which is characterized by the replacement of functional tissue with a large amount of fibrotic tissue produced by extensive connective tissue hyperplasia and extracellular matrix deposition. Studies have shown that fibrosis is the main cause of disability and death from many chronic diseases. Many COVID-19 patients have post-inflammatory pulmonary fibrosis after discharge from the hospital, which greatly affects the patient's prognosis and quality of life. Currently in clinical practice, although a few drugs or cell therapy means can be used to alleviate the process of fibrosis in specific organs and specific types, they cannot restore damaged tissues. And there is an extreme lack of broad and effective anti-fibrosis means. Therefore, there is an urgent need to develop broad-spectrum anti-fibrosis and regeneration-promoting candidate targets and drugs. Fibrosis is a non-regenerative repair manner for damage. Promoting the regeneration of damaged tissue (regenerative therapy) can not only control fibrosis, but also restore the original function of the tissue. Therefore, regenerative therapy is the most ideal means to prevent and treat fibrosis-related diseases.

Pulmonary fibrosis is a pathological change characterized by the proliferation of fibroblasts and the accumulation of a large amount of extracellular matrix, accompanied by inflammatory damage and tissue structure destruction. That is, normal alveolar tissue is damaged and then repaired abnormally, resulting in structural abnormalities (scar formation). Pulmonary fibrosis can seriously affect the human respiratory function, manifesting as various breathing difficulties that worsen with the worsening of the condition and lung damage, as well as the decline of the patient's respiratory function. It is reported that the incidence and mortality of idiopathic pulmonary fibrosis are increasing year by year worldwide, and the average survival period after diagnosis is less than 3 years, which is higher than most tumors. Therefore, it is also called a “tumor-like disease.” Therefore, promoting the regeneration of damaged tissue is the most fundamental means to treat and prevent diseases related to pulmonary fibrosis and has important application value.

### SUMMARY

In order to achieve the regeneration and repair of mammalian tissues and organs and to develop more technical approaches for the prevention and treatment of related diseases, the present application provides a small molecule compound with the capacity to promote the regeneration and repair of mammals, which has achieved groundbreaking and unexpected technical effects. The technical solution of this application is as follows: The present application provides a substance capable of up-regulating ISG gene expression, and use of the substance capable of up-regulating ISG gene expression in the promotion of regeneration and repair capacity of a tissue, complex structure or organ of a mammal. Furthermore, the substance capable of up-regulating the ISG gene expression is any one or more selected from the group consisting of: a MAPK inhibitor, retinoic acid receptor-related orphan receptor inhibitor, protein synthesis inhibitor, interferon (IFNγ, β, λ) and alarmin (S100A8/A9) protein.

ISG genes refer to the STING-TBK1-IRF3-interferon-stimulated gene (ISG) pathway. After interferon (IFN) acts on the surface receptors of target cells, it activates the expression of interferon-stimulated genes through a series of signal transduction. Interferon-stimulated genes and their expression products have multiple biological functions such as antiviral and immune regulation, and are important effector molecules for interferon to function.

Furthermore, the promotion of regeneration and repair capacity of the tissue, complex structure or organ of the mammal described in the present application is achieved by inducing activation of the TBK1-IRF3 pathway, preferably, by inhibiting protein synthesis.

The present application also provides use of a substance capable of up-regulating ISG gene expression in the preparation of a medicament or reagent for promoting regeneration and repair capacity of a tissue, complex structure or organ of a mammal.

The present application also provides use of a substance capable of up-regulating ISG gene expression in the preparation of a medicament or reagent for treating a disease related to regeneration and repair of a tissue, complex structure or organ of a mammal.

Furthermore, the regeneration and repair described in the present application refers to promotion of the regeneration of a tissue, complex structure or organ after tissue or organ resection or damage.

Preferably, the tissue described in the present application is skin, fat, muscle, bone, hair follicle, blood vessel or nerve.

Preferably, the complex structure described in the present application is a body structure including at least two or more selected from the group consisting of: skin, hair follicle, gland, cartilage, muscle, fat, blood vessel, nerve, and limb.

Preferably, the organ described in the present application is lung, liver, heart, pancreatic islet or kidney.

Further preferably, the complex structure is an ear, a limb, a finger, an eye or a nose.

Preferably, the regeneration and repair described in the present application is regeneration after ear resection.

Preferably, the regeneration and repair described in the present application is promotion of regeneration and repair after skin damage, hair regeneration after hair loss, regeneration and repair of cartilage and muscle damage, regeneration of fibrosis of lung, liver, skin, heart, kidney and muscle, and regeneration after blood vessel, nerve and limb damage.

Preferably, the regeneration and repair described in the present application is to promote regeneration and repair of scalded skin.

Preferably, the disease related to the regeneration and repair of the tissue or complex structure or organ described in the present application is skin scald, skin trauma, skin burn, hair loss, cartilage and muscle damage, liver fibrosis, pulmonary fibrosis or limb damage.

The MAPK inhibitor provided in the present application is used to promote the regeneration and repair capacity of a tissue and organ by inhibiting the p38MAPK pathway. Preferably, this is achieved by inhibition of P38δ.

The MAPK inhibitor provided in the present application may be any one or more selected from the group consisting of: a P38 inhibitor and a selective P38δ inhibitor. Preferably the P38 inhibitor is Doramapimod, and the selective P388 inhibitor is MAPK13-IN-1.

The retinoic acid receptor-related orphan receptor inhibitor provided in the present application is used to promote the regeneration and repair capacity of a tissue and organ by reversely activating the retinoic acid receptor-related orphan receptor α (RORα).

The retinoic acid receptor-related orphan receptor inhibitor provided in the present application may be a selective RORα inverse agonist. Preferably, it may be SR3335.

The protein synthesis inhibitor provided in the present application is used to promote the regeneration and repair capacity of a tissue and organ by inducing life quiescence.

The protein synthesis inhibitor provided in the present application is used to promote the regeneration and repair capacity of a tissue and organ by activating the STING-TBK1-IRF3 signal.

The protein synthesis inhibitor provided in the present application can be any one or more selected from the group consisting of: cycloheximide (CHX), anisomycin (Ani), didemnin B (DIDB), bouvardin (BVD), narciclasine and pancratistatin.

The present application also provides the use of a composition containing the substance capable of promoting the regeneration and repair capacity of a tissue, complex structure or organ of a mammal, in the promotion of regeneration and repair capacity of a tissue, complex structure or organ of a mammal, in the preparation of a medicament or reagent for promoting the regeneration and repair capacity of a tissue, complex structure or organ of a mammal, and in the preparation of a medicament or reagent for treating a disease related to regeneration and repair of a tissue, complex structure or organ of a mammal.

Preferably, the composition provided by the present application comprises a protein synthesis inhibitor, and an all-trans retinoic acid and a BMP activator.

Preferably, the protein synthesis inhibitor in the composition is cycloheximide (CHX), and the BMP activator is BMP signaling agonist sb4.

Preferably, in the composition, based on 1 part by weight of the cycloheximide, the amount of the all-trans retinoic acid is 0.25 to 8 parts by weight, and the amount of the BMP signaling agonist sb4 is 0.25 to 4 parts by weight.

The present application also provides that the administration method of the substance capable of promoting capacity of regeneration and repair of a tissue, complex structure or organ of a mammal or a composition thereof can be intraperitoneal injection, intravenous injection, oral gavage, oral administration or skin application.

The present application also provides a method for promoting regeneration and repair of a tissue, complex structure or organ of a mammal, comprising administering a substance capable of up-regulating ISG gene expression or a composition comprising the substance capable of up-regulating the ISG gene expression to a subject in need thereof.

Furthermore, the substance capable of up-regulating ISG gene expression or the composition comprising the substance capable of up-regulating ISG gene expression in the method is the substance capable of up-regulating ISG gene expression provided in the present application or the composition provided in the present application.

Furthermore, in the method, the substance capable of up-regulating ISG gene expression or the composition comprising the substance capable of up-regulating ISG gene expression can be administered to a subject in need thereof by an administration manner, such as intraperitoneal injection, intravenous injection, oral gavage, oral administration or skin application.

### BRIEF DESCRIPTION OF THE DRAWINGS

FIG. 1A shows the differential gene analysis and functional enrichment results between the transcriptomes of regenerative African agouti and non-regenerative mice.

FIG. 1B is a schematic diagram of the healing of a 2 mm diameter mouse ear hole after treated with Vehicle and different protein translation inhibitors Ani and CHX drugs.

FIG. 1C is a schematic diagram of the closure of a 2 mm diameter mouse ear hole after treatment with Vehicle and different doses of CHX drug.

FIG. 1D is a schematic diagram of the closure of a mouse ear hole injured by a 2 mm diameter ear puncher after 30 days of CHX drug treatment, the scale bar is 1 mm.

FIG. 1E shows the HE staining results of mouse auricle tissue after CHX drug treatment, and the scale bar is 200 um.

FIG. 1F shows the HE staining results of mouse auricle tissue after CHX drug treatment, and the scale bar is 1 mm.

FIG. 1G is a schematic diagram of the KI67 immunohistochemical staining results of the mouse auricle tissue after 7 days of CHX drug treatment, and the scale bar is 100 μm.

FIG. 1H is a schematic diagram of the HE staining results of the mouse auricle tissue 180 days after the ear hole was closed by CHX drug treatment, and the scale bar is 1 mm.

FIG. 1I is a schematic diagram of the healing effect of a mouse ear hole after 3 weeks of treatment with different drug administration manners (21 days after damage).

FIG. 1J shows the healing effect of 2 mm ear holes in Nsun2 knockout mice (21 days after damage).

FIG. 2A is a schematic diagram of the closure of a 4 mm diameter mouse ear hole after treatment with DMSO/CHX.

FIG. 2B is a schematic diagram of the closure of a mouse ear hole injured by a 4 mm diameter ear puncher after 50 days of CHX treatment, and the scale bar is 1 mm.

FIG. 3 shows the healing effect of mouse ear holes treated with inhibitors of protein and RNA synthesis, ferroptosis and autophagy (21 days after damage).

FIG. 4A is a schematic diagram of the analysis of genes that are co-regulated at the transcriptional and translational levels by CHX.

FIG. 4B is a schematic diagram of interferon response genes and their enrichment.

FIGS. 4C and 4D are schematic diagrams of the experimental results of quantitative qPCR detection of CHX-induced ISG gene expression.

FIG. 4E is a schematic diagram of the results of immunofluorescence staining to identify CHX activating the STING/TBK1/IRF3 signaling pathway.

FIG. 4F is a schematic diagram of the results of quantitative qPCR detection of GSK86126 and Des inhibiting CHX-induced ISG gene expression by inhibiting the STING/TBK1/IRF3 pathway activity.

FIG. 4G is a schematic diagram of the results of GSK86126 and Des inhibiting CHX-induced regeneration by inhibiting the STING/TBK1/IRF3 pathway activity.

FIG. 4H is a schematic diagram of the results of a Western blot experiment revealing that Des does not inhibit the CHX protein synthesis inhibition function by inhibiting the STING/TBK1/IRF3 pathway activity.

FIG. 5A is a schematic diagram of the experimental results of the Puromycin (PURO) incorporation experiment to verify the protein translation inhibition effect of narciclasine.

FIGS. 5B and 5C are schematic diagrams of the results of quantitative qPCR detection of narciclasine-induced ISG gene expression.

FIG. 5D is a schematic diagram of the closure of a mouse ear hole injured by a 2 mm diameter ear puncher after treatment with Nar drug for 30 days, and the scale bar is 1 mm.

FIG. 5E is a schematic diagram of the closure of a mouse ear hole with a diameter of 2 mm after treatment with Vehicle and different doses of Nar drug.

FIG. 5F is a schematic diagram of the immunohistochemistry results after treatment with Nar drug.

FIG. 5G is a schematic diagram of the Masson section staining results after treatment with Nar drug.

FIG. 5H is a schematic diagram of the healing of a mouse ear hole with a diameter of 4 mm after treatment with Vehicle/NRB.

FIG. 5I is a schematic diagram of the immunohistochemistry results after treatment with NRB.

FIG. 5J is a schematic diagram of HE section staining results after treatment with NRB.

FIG. 6A is a schematic diagram of the experimental results of the Puromycin incorporation experiment to verify the protein translation inhibitory effect of pancratistatin.

FIGS. 6B and 6C are schematic diagrams of the experimental results of quantitative qPCR detection of pancratistatin induced ISG gene expression.

FIG. 6D is a schematic diagram of the closure of a mouse ear hole injured by a 2 mm diameter ear puncher after treatment with Pan drug for 21 days, and the scale bar is 1 mm.

FIG. 6E is a statistical diagram of the effect of Pan treatment on a mouse ear hole injured by a 2 mm diameter ear puncher for 21 days, with DMSO replacing Pan as the control.

FIG. 6F is a schematic diagram of the HE slice results after treatment with Pan drug.

FIG. 6G is a schematic diagram of the HE slice results after treatment with Pan drug.

FIGS. 7A and 7B are schematic diagrams of the experimental results of quantitative qPCR detection of interferon (IFN)γ-induced ISG gene expression.

FIG. 7C is a statistical diagram of the effect of interferon (IFN)γ treatment for 35 days on a mouse ear hole injured by a 2 mm diameter ear puncher.

FIGS. 8A and 8B are schematic diagrams of the experimental results of quantitative qPCR detection of S100A8/A9-induced ISG gene expression.

FIG. 8C is a statistical diagram of the effects of S100A8/A9 treatment for 35 days on a mouse ear hole injured by a 2 mm diameter ear puncher.

FIG. 8D shows HE staining (left) and Masson staining (right) results of a mouse ear hole injured by a 2 mm diameter ear puncher treated with S100A8/A9 drug.

FIG. 9A is a schematic diagram of the effects of different MAPKs inhibitors on promoting ear hole healing. The results show that P38 or P38δ inhibitors significantly promote regeneration.

FIG. 9B is a schematic diagram of the experimental results of the quantitative qPCR detection of MAPK13-IN-1-induced ISG gene expression.

FIG. 9C is the HE staining results of MAPK13-IN-1-induced regeneration of auricle tissue.

FIG. 9D is the Masson staining result of MAPK13-IN-1-induced regeneration of auricle tissue.

FIG. 10A is a schematic diagram of the effects of RORα inverse agonist (inhibitor) and agonist on promoting ear hole healing.

FIGS. 10B and 10C are schematic diagrams of the experimental results of quantitative qPCR detection of SR3335-induced ISG gene expression.

FIG. 10D is the HE staining results of SR3335-induced regeneration of auricle tissue.

FIG. 10E is the Masson staining result of SR3335-induced regeneration of auricle tissue.

FIG. 10F is the HE staining result of the regenerated auricle tissue of Rora homozygous knockout mice.

FIG. 11A is a schematic diagram of the healing of the mouse ear holes after treatment with Vehicle/CRB.

FIG. 11B is a diagram of the healing effect of a 4 mm mouse ear hole after treatment with Vehicle/CRB for 30 days.

FIG. 11C is a HE staining image of the mouse auricle tissue after treatment with Vehicle/CRB.

FIG. 11D is an immunofluorescence staining image of α-SMA of mouse auricle tissue after treatment with Vehicle/CRB for 7 days.

FIG. 11E is a schematic diagram of HE staining results of mouse auricle tissue after the 4 mm diameter mouse ear hole was treated with Vehicle/CRB for more than 180 days, and the scale bar is 100 μm.

FIG. 11F is the result of immunofluorescence staining of vascular marker CD31, and the scale bar is 20 μm.

FIG. 12A is a schematic diagram of limb modeling.

FIG. 12B shows the phenomenon of limb regeneration induced by drug treatment for 20, 40, and 120 days, and the scale bar is 2 mm.

FIG. 12C is the statistical result of the outgrowth length of the limbs. n>3, *** p<0.001, t-test.

FIG. 12D shows a high-definition image of the bone tissue reconstruction process obtained by electronic computed tomography (CT) after 20 days and 40 days of drug CR treatment.

FIG. 12E shows the regeneration results of bone tissue after 30 days of drug CR treatment.

FIG. 12F shows HE staining showing that the combination can promote the regeneration of multiple tissue types at the amputated limb.

FIG. 12G shows the results of Masson staining of mouse limb regeneration tissue after 21, 50, and 120 days of drug treatment, and the scale bar is 2 mm, n≥3, t-test.

FIG. 13 is a schematic diagram of activating ISG gene expression to promote mammalian regeneration ability, and to promote regeneration and repair.

### DETAIL DESCRIPTION

The following is an elaboration and description of the embodiments of the present invention through specific examples, but the following contents should not be construed as limiting the present invention in any way.

The present application mainly relates to a substance capable of promoting regeneration and repair capacity of a tissue, complex structure or organ of a mammal, wherein the substance is a substance capable of up-regulating ISG gene expression.

In the present application, the substances capable of up-regulating the ISG gene expression include compounds, cytokines, proteins and inhibitors.

In a specific embodiment, the substance capable of up-regulating the ISG gene expression is any one or more selected from the group consisting of: MAPK inhibitors, retinoic acid receptor-related orphan receptor inhibitors, protein synthesis inhibitors, interferons (IFNγ, β, λ) and alarmin (S100A8/A9) proteins.

The present application also relates to use of a substance capable of up-regulating the ISG gene expression in the preparation of a medicament or reagent for promoting regeneration and repair capacity of a tissue, complex structure or organ of a mammal.

The present application also relates to use of a substance capable of up-regulating the ISG gene expression in the preparation of a medicament or reagent for treating a disease related to regeneration and repair of a tissue, complex structure or organ of a mammal.

In an embodiment of the present application, the promotion of the regeneration and repair capacity of the tissues, complex structures or organs of mammals is achieved by inducing activation of a TBK1-IRF3 pathway.

In a specific embodiment of the present application, the substance capable of up-regulating the ISG gene expression is a MAPK inhibitor. A MAPK (mitogen-activated protein kinase) pathway has three levels of signal transduction: MAPK, MAPK kinase (MEK or MKK) and MAPK kinase kinase (MEKK or MKKK). These three kinases can be activated in sequence to jointly regulate many important physiological and pathological effects, such as cell growth, differentiation, stress, and inflammatory response. There are four main branches of the MAPK pathway: ERK, JNK, p38/MAPK and ERK5. Among them, ERK regulates cell growth and differentiation, and the JNK and p38MAPK signaling pathways play an important role in stress responses such as inflammation and cell apoptosis. The MAPK inhibitors can regulate signal transduction of various MAPK pathways.

In a specific embodiment of the present application, the promotion of regeneration and repair capacity of tissues and organs is achieved by inhibiting the p38MAPK pathway, and further, by inhibiting a P38δ pathway.

In some specific embodiments, the MAPK inhibitor is any one or more selected from a specific P38 inhibitor or a selective P38δ inhibitor. Preferably, the P38 inhibitor is Doramapimod, and the selective P38δ inhibitor is MAPK13-IN-1.

In a specific embodiment of the present application, the substance capable of up-regulating the ISG gene expression is a retinoic acid receptor-related orphan receptor inhibitor, specifically a retinoic acid receptor-related orphan receptor α (RORα) inhibitor.

In a specific embodiment of the present application, the promotion of the regeneration and repair capacity of the tissues and organs is achieved by reverse activation of the retinoic acid receptor-related orphan receptor α (RORα). In some specific embodiments, the RORα inhibitor is a selective RORα inverse agonist, preferably SR3335.

In a specific embodiment of the present application, the substance capable of up-regulating the ISG gene expression is a protein synthesis inhibitor. The protein synthesis inhibitors are a class of substances that affect protein biosynthesis. They can act on DNA replication and RNA transcription, and indirectly affect the protein biosynthesis. They can act on various links of the protein synthesis, including inhibiting effects of initiation factors, elongation factors, and ribonucleoproteins, etc. Among them, common protein synthesis inhibitors are mainly blockers that can inhibit a translation process of the protein biosynthesis.

In a specific embodiment of the present application, the promotion of the regeneration and repair capacity of the tissue and organ is achieved by inhibiting the protein synthesis.

In some specific embodiments, the protein synthesis inhibitor can be any one or more selected from the group consisting of: cycloheximide (CHX), didemnin B (DIDB), bouvardin (BVD), narciclasine and pancratistatin.

In a specific embodiment of the present application, the protein synthesis inhibitor is cycloheximide, abbreviated its chemical formula is: as CHX, and (4-((R)-2-((1S,3S,5S)-3,5-dimethyl-2-oxocyclohexyl)-2-hydroxyethyl)piperidine-2,6-dione).

The present application also provides use of a composition comprising the substance capable of up-regulating ISG gene expression in the promotion of regeneration and repair capacity of a tissue, complex structure or organ of a mammal, or in the preparation of a medicament or reagent for promoting regeneration and repair capacity of a tissue, complex structure or organ of a mammal, or in the preparation of a medicament or reagent for treating a disease related to regeneration and repair of a tissue, complex structure or organ of a mammal.

In a specific embodiment, the composition comprises a protein synthesis inhibitor, and an all-trans retinoic acid, and a BMP activator.

In a preferred embodiment, the protein synthesis inhibitor in the composition is cycloheximide

(CHX), and the BMP activator is BMP signaling agonist sb4.

In a preferred embodiment, in the composition, based on 1 part by weight of the cycloheximide, the amount of the all-trans retinoic acid is 0.25 to 8 parts by weight, and the amount of the BMP signaling agonist sb4 is 0.25 to 4 parts by weight.

In the embodiments of the present application, the regeneration and repair refers to the repair process in which a structure with the same morphology and function as the lost part grows on the basis of the remaining part after the whole body, an organ or local tissue of a mammalian organism is traumatized and partially lost.

In a specific embodiment, the regeneration and repair is promotion of the regeneration of tissues or complex structures or organs after tissue or organ resection or damage.

In some specific embodiments, the tissue is epidermis, dermis, muscle, bone, fat, hair follicle, blood vessel or nerve.

In some specific embodiments, the complex structure is at least two or more selected from the group consisting of: body structure comprising skin, hair follicle, gland, cartilage, muscle, fat, blood vessel, nerve, and limb.

In some specific embodiments, the organ is lung, skin, heart, liver, kidney, stomach, intestine, etc.

The complex structures described in this application are body structural parts composed of different tissues or body functional parts that can perform specific physiological functions or functional activities, such as ears, organs, limbs, eyes, nose, etc.

In a preferred embodiment, the complex structure is an ear hole.

In a specific embodiment, the regeneration and repair is promotion of regeneration after a partial ear resection.

In a specific embodiment, the regeneration and repair is promotion of regeneration and repair after skin damage, hair regeneration after hair loss, regeneration and repair of cartilage and muscle damage, regeneration of lung, liver, skin, heart, kidney, muscle fibrosis, and regeneration after blood vessel, nerve and limb damage.

In a specific embodiment, the regeneration and repair is promotion of regeneration and repair of scalded skin.

In an embodiment of the present application, the diseases related to the regeneration and repair of tissues and organs include but are not limited to, skin burns, organ fibrosis, muscle/cartilage damage or nervous system diseases, and are preferably skin burns/scalds/trauma, hair loss, cartilage and muscle damage, pulmonary fibrosis, liver fibrosis, kidney fibrosis, myocardial fibrosis, limb trauma or various nervous system diseases.

In the embodiments of the present application, pharmaceutically acceptable carriers or excipients may also be added to the medicament or reagent.

Specifically, the medicament or reagent can be prepared in the following form: the protein synthesis inhibitor or a composition comprising the same is mixed with the pharmaceutically acceptable carrier to obtain, for example, oral preparations, such as tablets (including sugar-coated tablets, film-coated tablets, sublingual tablets, orally disintegrating tablets), capsules (including soft capsules, microcapsules), granules, powders, lozenges, syrups, emulsions, suspensions, films (for example, orally disintegrating films), etc., parenteral preparations such as injections (for example, subcutaneous injections, intravenous injections, intramuscular injections, intraperitoneal injections, drops), external preparations (for example, skin preparations, ointments), suppositories (for example, rectal suppositories, vaginal suppositories), pills, nasal drops, respiratory preparations (inhalants), eye drops, etc. Besides, these preparations can be used as controlled release preparations (e.g., sustained release microcapsules), such as immediate release preparations, sustained release preparations, etc. Such preparations can be obtained by preparation methods conventionally used in the art.

Specifically, examples of the above-mentioned pharmaceutically acceptable carriers include excipients (e.g., starch, lactose, sucrose, calcium carbonate, calcium phosphate, etc.), binders (e.g., starch, gum arabic, carboxymethylcellulose, hydroxypropylcellulose, crystalline cellulose, alginic acid, gelatin, polyvinylpyrrolidone, etc.), lubricants (e.g., magnesium stearate, calcium stearate, talc, etc.), disintegrants (e.g., carboxymethylcellulose calcium, talc, etc.), diluents (e.g., water for injection, saline, etc.), additives (e.g., stabilizers, preservatives, colorants, flavorings, dissolution aids, emulsifiers, buffers, isotonic agents, etc.), and the like.

In an embodiment of the present application, the substance, composition, medicament or reagent capable of up-regulating the ISG gene expression may be administered by intraperitoneal injection, intravenous injection, oral administration or skin application. Specifically, the dosage to be administered to a subject varies depending on the administration route, symptoms, patient age, etc., and can be determined practically by a clinician.

### EXAMPLES

Studies in recent years have shown that MRL mice 1 and P21-/- mutant mice 2 can close 2 mm (millimeter) ear holes, while wild-type experimental mice cannot. Therefore, ear hole closure can be used as a good model for assessing regeneration capacity to screen cells, signaling pathways, and genes that promote and improve regeneration capacity. The main screening targets are important signaling pathways that regulate morphogenesis, organ development, immunity and stress response (metabolism, translation, etc.) during individual development; they also comprise genes and signaling pathways involved in tumorigenesis and regeneration of lower organisms.

Establishment of ear hole trauma mouse model: 7-week-old C57BL/6 mice were selected and anesthetized with 5% chloral hydrate, with 10 mL/kg injected intraperitoneally according to body weight. The anesthetized mice were tied up and the ears and instruments were disinfected with 75% ethanol. The left and right auricles of the mice were punched at the center of the auricle using a 2 mm diameter ear puncher. The traumatized mice were administered drugs (small molecules or growth factors for targeted screening targets dissolved in saline or DMSO) by intraperitoneal injection, intravenous injection or oral gavage, and the solvent without drugs was used as a control. The DMSO-dissolved drug delivery system was: 2-5% DMSO+30-40% PEG400+2-5% Tween 80 were added in sequence according to the final concentration (volume ratio). The drug was administered every 2 days and the mice were observed every 7 days. The proximal-distal diameter (DPD) and anterior-posterior diameter (DAP) of the mouse ear holes were measured using a vernier caliper, and the area of the mouse ear holes at excision was calculated using the area calculation formula S=πDPD×DAP/4. The closure of the mouse ear holes was recorded.

Unless otherwise specified, the ear hole trauma mouse model mentioned above was used in the following Examples of the present application for ear hole regeneration experiments, wherein 7-week-old C57BL/6 mice were purchased from Beijing Charles River Experimental Animal Technology Co., Ltd.; Nsun2 knockout mice were prepared by the applicant's laboratory, and CRISPR/Cas9-mediated gene knockout technology was used to inject Cas9 mRNA and Nsun2 sgRNA into mouse fertilized eggs to obtain gene knockout embryos, and further breeding obtained stable knockout mice; CHX: (i.e., 4-((R)-2-((1S,3S,5S)-3,5-dimethyl-2-oxocyclohexyl)-2-hydroxyethyl)piperidine-2,6-dione) was purchased from MedCamExpress. Other materials and reagents, unless otherwise specified, can be obtained from commercial sources.

### Example 1: Translation Inhibition (Cycloheximide (Hereinafter Abbreviated as CHX or C); Anisomycin (Hereinafter Abbreviated as Ani)) Promotes the Regeneration at Excision of 2 mm Diameter of Mouse Ear Holes

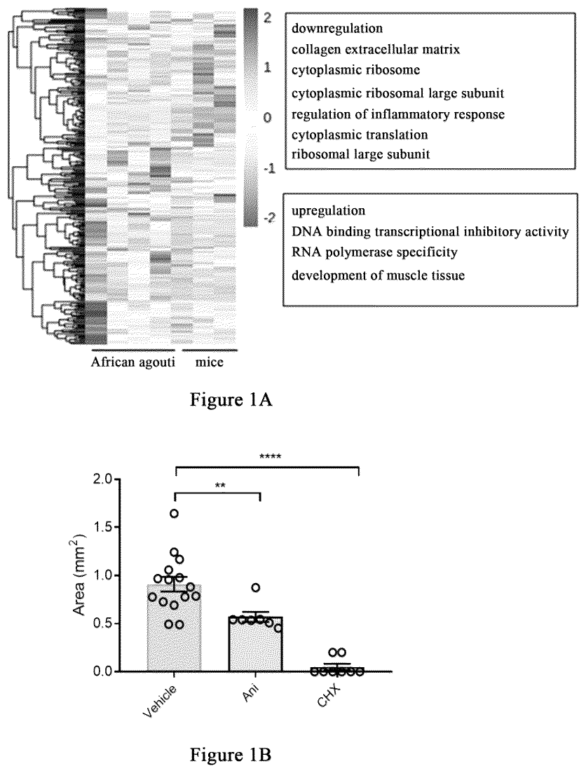

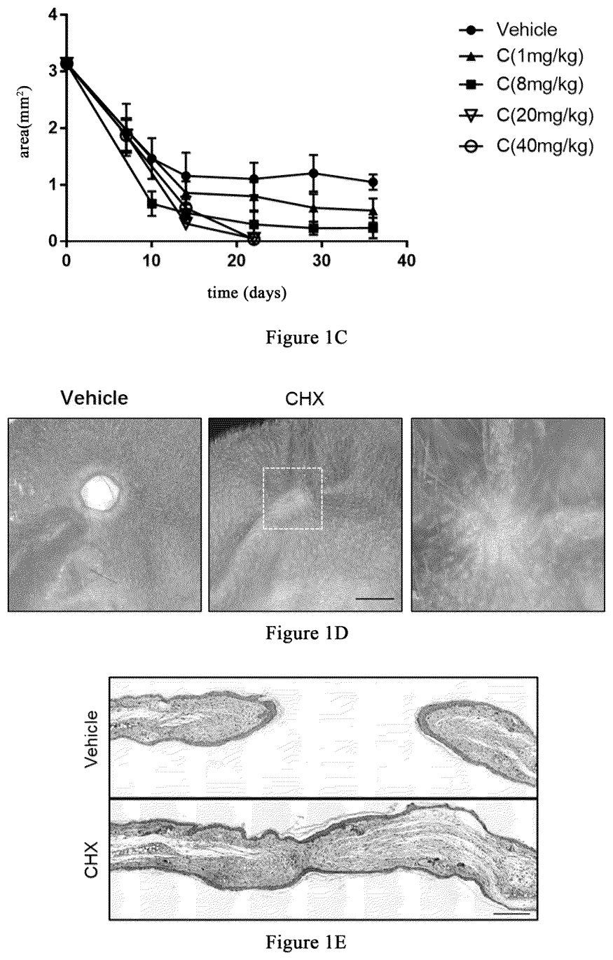

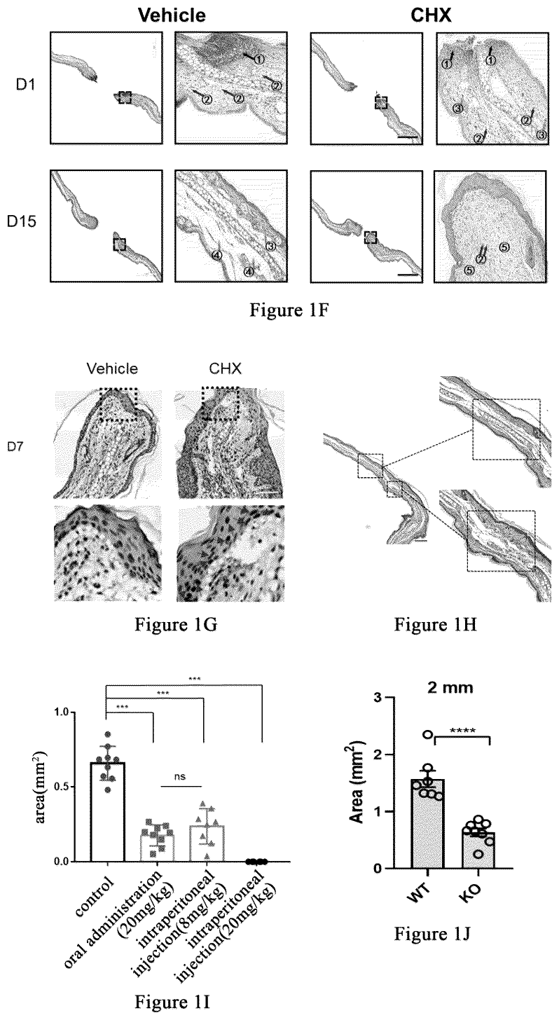

Experimental method: Different doses of CHX (dissolved in saline or DMSO) were intraperitoneally injected into mice with ear hole trauma (in the experiment of FIG. 1I, oral gavage administration was added), and saline or DMSO served as the control group (marked as Vehicle). Identification of traumatic auricles of drug-treated mice was performed. The specific experimental results were shown in FIG. 1.

FIG. 1A showed transcriptome analysis of differentially expressed genes between regenerative African agouti and non-regenerative mice (C57BL/6 mice), and functional enrichment analysis showed that the genes significantly downregulated in regenerative African agouti compared with non-regenerative mice were ribosomal subunits and translation-related.

FIG. 1B showed that based on the analysis results of FIG. 1A, non-regenerative mice were treated with small molecule inhibitors of translation Ani and CHX for 21 days respectively, and the healing effect on the 2 mm of ear hole was observed. The results showed that compared with the control group (Vehicle), the translation inhibitors Ani (10 mg/kg) and CHX (20 mg/kg) could significantly promote the healing of the ear hole, and the healing effect of CHX was better. It was found that translation inhibition could significantly promote the regeneration of non-regenerative species. Subsequent experiments were carried out based on CHX.

The schematic diagram of FIG. 1C showed the closure of mouse ear holes after treatment with different concentrations of Vehicle/CHX: each concentration of CHX promoted the closure of a 2 mm diameter of ear holes, and when the concentration was greater than 8 mg/kg, a wound of the mouse ear hole was completely closed after three weeks of drug treatment. n≥8.

FIG. 1D was a photograph showing the healing condition of a 2 mm ear hole of mice with ear hole trauma treated with Vehicle/CHX (20 mg/kg) 30 days later, in which the ear hole wound treated with CHX had completely closed.

The HE staining image of the mouse auricle tissue in FIG. 1E showed that after treatment with CHX (20 mg/kg), the skin, connective tissue and other tissue structures in the ear hole of the mouse with ear hole trauma grew and connected together, and the trauma had completely healed.

The HE staining image of the mouse auricle tissue in Figure IF showed that after the mice with ear hole trauma were treated with DMSO/CHX (20 mg/kg), focal necrosis was locally observed in both tissues on the first day after trauma (D1 group), and inflammatory cells diffusely infiltrated in the necrotic focus, as shown by arrow \textcircled{1}; diffuse infiltration of inflammatory cells was observed in the dermis of the tissue, as shown by arrow \textcircled{2}; among them, hemosiderin deposition was observed in some cells in the CHX group, as shown by arrow \textcircled{3}. On the 15th day, hemosiderin deposition was observed in some cells of the tissues of the DMSO group, as indicated by arrow \textcircled{3}; tissue edema was observed, the spaces between subdermal connective tissue were enlarged, and the tissue structure was loose, as shown by arrow \textcircled{4}. On the 15th day, the CHX group healed the tissue after damage and formed granulation tissue. Fibroblasts and blood vessels proliferated in large numbers in the granulation tissue, as shown by arrow 5. In addition, the inflammatory cells were diffusely distributed, as shown by arrow \textcircled{2}.

The KI67 immunohistochemical staining image of mouse auricle tissue in FIG. 1G showed that after 7 days of CHX (20 mg/kg) treatment, the basal cells of mice expressed a large amount of KI67, a marker protein of cell proliferation, as shown by the arrow, while the expression in the control group was relatively low.

The HE staining image of the mouse auricle tissue in FIG. 1H showed that 180 days after the ear holes of mice treated with CHX (20 mg/kg) were closed, the wound site showed regeneration of tissues and tissue derivatives such as hair follicles, glands, cartilage, muscles and blood vessels.

The schematic diagram of FIG. 1I showed that different administration manners such as oral gavage and intraperitoneal injection were used to treat mice with ear hole trauma, and healing effects were produced after 3 weeks (21 days after damage). Wherein n≥6. ***p<0.001, ns: no significant difference, t-test.

In addition, Nsun2 knockout had been reported to inhibit translation by regulating tRNA stability, so Nsun2 knockout mice were used as a genetic model to verify the effect of translation inhibition on regeneration. The specific method is: ear hole trauma mouse models were established using wild-type mice (WT) and Nsun2 knockout mice (KO), 2 mm diameter of ear holes were made (the method is the same as the above Examples), and the healing condition of the ear holes was observed and measured after 3 weeks. The results showed that the ear hole area of the Nsun2 knockout mice was significantly reduced compared with that of the wild-type mice. This also suggested that translational inhibition had a role in regeneration (FIG. 1J).

The above experimental results showed that different doses of protein synthesis inhibitor CHX could significantly promote the healing of 2 mm ear hole, and this promoting effect was dose-dependent. Doses greater than 8 mg/kg could promote closure. The closure of the ear hole could regenerate tissue and tissue derivatives such as hair follicles, glands, cartilage and muscle. It was also confirmed that different manners of administration had the effect of promoting regeneration.

### Example 2: Cycloheximide CHX Promotes the Regeneration and Repair of 4 mm Diameter of Mouse Ear Hole

The study found that very few mammals, such as the African agouti, have strong regeneration capacities and could regenerate a 4 mm ear hole. It also found that a closure model of the 4 mm ear hole could distinguish between strong and weak regeneration capacities. Currently, there was no artificial way to achieve closure and regeneration of the 4 mm ear hole. Even the MRL mice reported to have “super regeneration” could not completely close the hole. Therefore, the 4 mm of ear hole could be used as a good model for evaluating the regeneration at excision in mammals. The model was established using the method of Example 1, in which the left and right auricles of mice were punched respectively with a 4 mm diameter ear puncher.

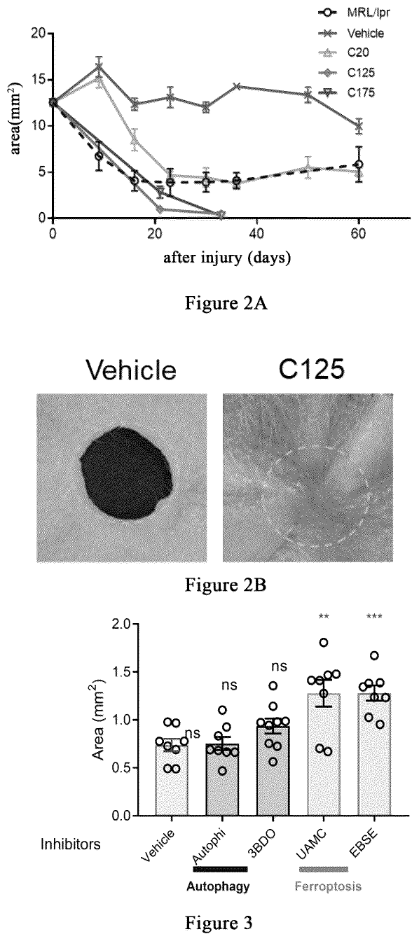

Experimental method: Different doses of CHX (20, 125, 175 mg/kg, dissolved in DMSO or saline) administered by intraperitoneal injection to mice with the 4 mm ear hole trauma. DMSO served as the control group. Identification of traumatic auricles of drug-treated mice was performed. The specific experimental results are shown in FIG. 2.

The schematic diagram of FIG. 2A showed the effect of different doses of CHX on the closure of 4 mm diameter ear hole. It was found that 20 mg/kg had a similar effect to that of super-healing mice MRL/lpr, which promoted the shrinkage of ear hole wounds, but could not completely close them. When the dose was greater than 20 mg/kg (125, 175 mg/kg), complete closure of the ear holes could be achieved.

FIG. 2B was a photograph of healing of the ear hole after treatment with DMSO/CHX drug for 90 days, which shows that the wound area of the 4 mm of mouse ear hole treated with CHX (125 mg/kg) was significantly reduced.

### Example 3: Cycloheximide (CHX) Promotes Ear Hole Regeneration Independently of the Inhibition of Ferroptosis and Autophagy

As an antifungal antibiotic, CHX could inhibit ferroptosis and cellular autophagy in addition to inhibiting eukaryotic protein synthesis and RNA synthesis. In order to verify which target it acts through during the regeneration process, small molecule validation was performed targeting ferroptosis and autophagy respectively. Experimental methods: The same ear hole trauma mouse model was used for the experiment, and inhibitors of ferroptosis and autophagy were used instead of CHX to observe their effects on promoting the healing of the ear hole. The control group in which DMSO replaced the small molecule. Auto: Autophinib (MCE, HY-101920), inhibits autophagy; 3BDO: 3BDO (MCE, HY-U00434), inhibits autophagy; UAMC: UAMC-3203 (MCE, MCE, HY-112909A), inhibits ferroptosis; EBSE: Ebselen (MCE, HY-13750), inhibits ferroptosis. The concentration used was 10-20 mg/kg, and the administration manner was the same as CHX, n≥8, **p<0.01, ***p<0.001, ns: no significant difference, t-test. Identification of the effects of different inhibitors on the healing of 2 mm ear holes was performed (measured 21 days after damage). The results were shown in FIG. 3: Neither inhibitors of ferroptosis nor autophagy could promote the healing of the ear hole like CHX, which indicates that CHX's promotion of ear hole regeneration is not dependent on its inhibitory activity on ferroptosis or autophagy.

### Example 4: Cycloheximide (CHX) Activates the STING-TBK1-IRF3-Interferon-Stimulated Genes (ISGs) Pathway, and the ISG Gene Expression is Necessary for Regeneration Induced by CHX

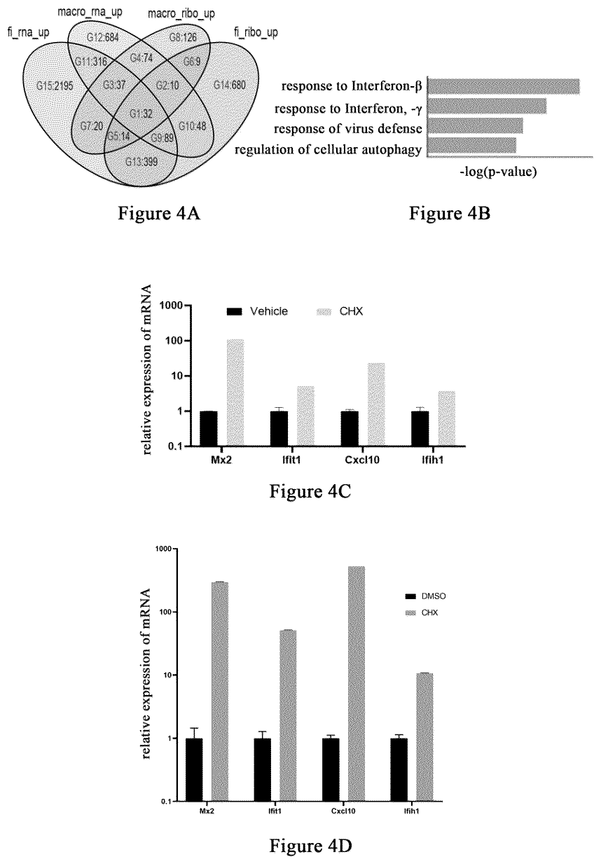

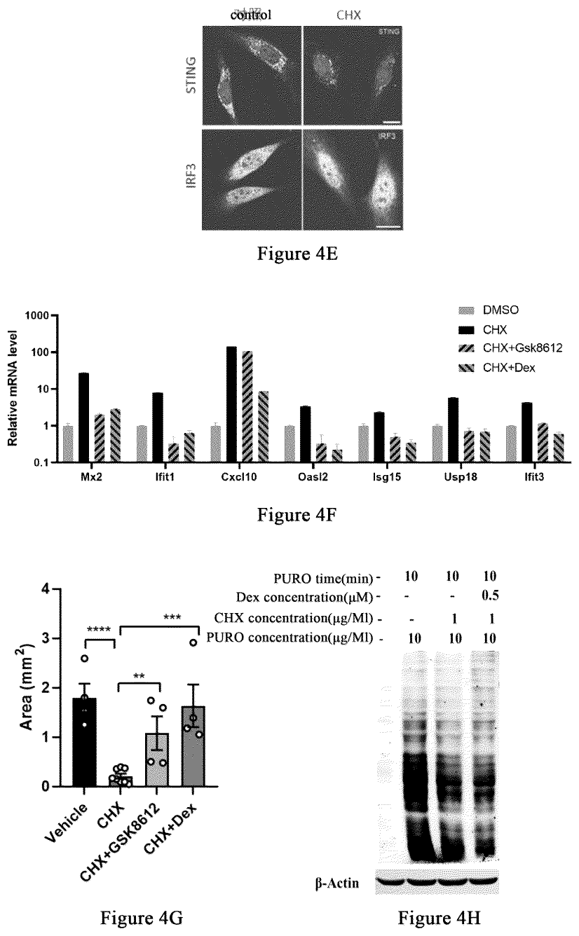

To further verify the mechanism of CHX-induced regeneration, mouse primary fibroblasts and macrophages were treated with CHX, and large-scale RNA-seq (transcription level) and Ribo-seq (ribosomal imprint sequencing, translatomics) were performed. Bioinformatics analysis revealed that CHX caused 32 genes to be upregulated at both the transcriptional and translational levels in both types of cells (G1, FIG. 4A). Functional analysis revealed that these genes were interferon-responsive genes, and their functions were mainly enriched in responses to interferon and viruses (FIG. 4B). qPCR experiments further verified that CHX could indeed upregulate the expression of classical ISGs, such as Mx2, Ifit1, Cxcl10, Ifih1, etc. (FIG. 4C showed the expression of fibroblast ISG genes, and FIG. 4D showed the expression of macrophage ISG genes). To verify whether the ISG gene expression is required for CHX-induced regeneration, we first found in vitro that CHX activated ISGs by activating the STING-TBK1-IRF3 signaling pathway, which was mainly reflected in the perinuclear enrichment of STING protein and the nuclear entry of IRF3 protein (FIG. 4E). Inhibition of TBK1 using small molecule inhibitors GSK8612 (5 μM) or Dexamethasone (Dex, 10 μM) significantly inhibited the expression of ISG genes induced by CHX (1 μg/ml) (FIG. 4F). In vivo, inhibition of TBK1 (GSK8612 (25 mg/kg), Dexamethasone (Dex, 10 mg/kg)) significantly inhibited CHX (20 mg/kg)-induced ear hole regeneration (FIG. 4G). In addition, through the Puromycin (PURO) incorporation experiment (see reference “Kearse, et al. Kearse, et al. Ribosome queuing enables non-AUG translation to be resistant to multiple protein synthesis inhibitors, 2019, Genes & Development”), it was found that Dexamethasone did not affect the inhibition of CHX on overall translation, indicating that CHX-mediated translation inhibition was located upstream of the induced ISG expression (FIG. 4H). The above indicated that the ISG gene expression was necessary for CHX-induced regeneration.

### Example 5: Narciclasine Activates ISG to Promote Regeneration in Mice

### (1) Narciclasine Activates the ISG Gene Expression

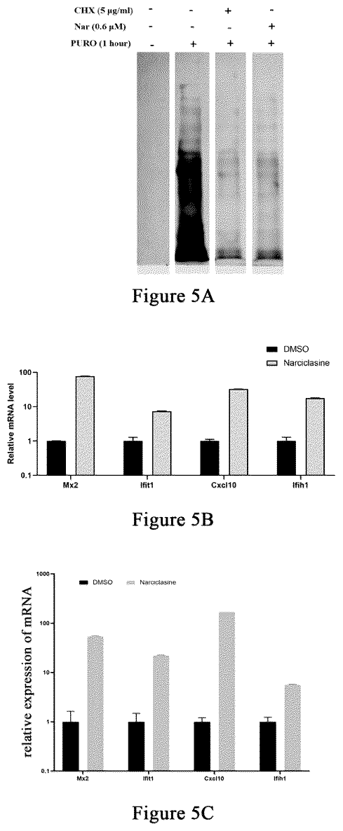

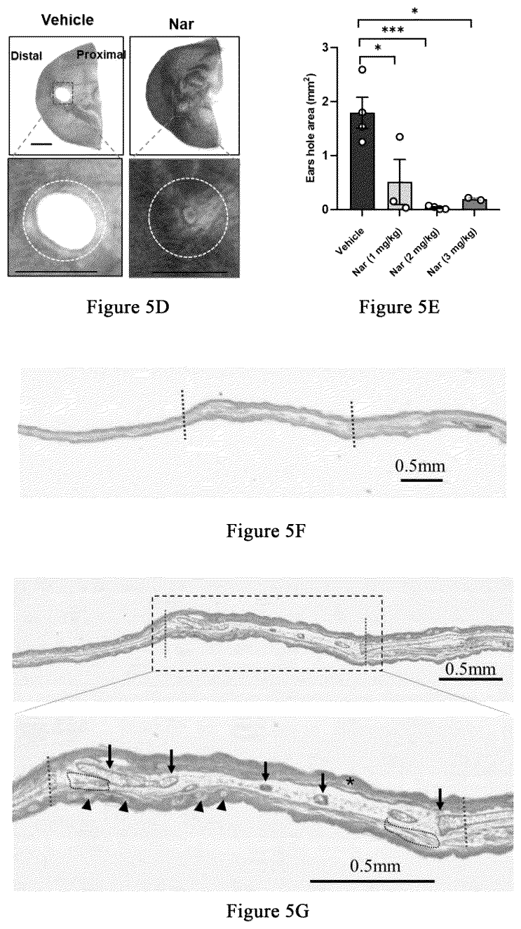

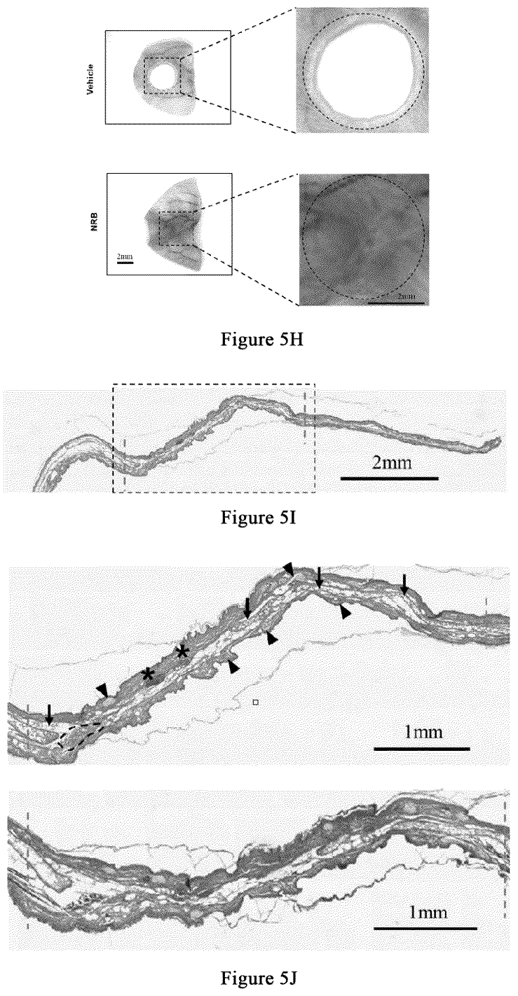

Narciclasine exists in various Amaryllidaceae plants and has extension and inhibitory effects of translation. First, through the Puromycin (PURO) incorporation experiment was conducted to verify that it could significantly inhibit the overall protein translation, as shown in FIG. 5A. Further quantitative qPCR revealed that it significantly promoted the expression of ISG genes, as shown in FIGS. 5B and 5C, wherein FIG. 5B showed the expression of ISG genes in fibroblasts, and FIG. 5C showed the expression of ISG genes in macrophages.

### (2) Narciscycline Promotes Regeneration of a 2 mm Mouse Ear Hole

Experimental method: Different doses (1-3 mg/kg) of Narciclasine (dissolved in DMSO, administration system: 2-5% DMSO +30-40% PEG400+2-5% Tween 80+saline) were administered by intraperitoneal injection to mice with 2 mm of ear hole trauma. DMSO without drug was used as the control group (labeled as Vehicle). Identification of traumatic auricles of drug-treated mice was performed. The specific experimental results were shown in FIGS. 5D to 5G.

Among them, FIG. 5D is a schematic diagram of the healing of the mouse ear holes injured by a 2 mm diameter ear puncher after treatment with Narciclasine for 30 days. FIG. 5E showed the regeneration effect of different doses of Narciclasine on mouse ear holes. FIGS. 5F and 5G were histochemical and Masson section staining data, respectively, characterizing the regeneration structures of cartilage (long black arrows), hair follicles (asterisks), glands/sebaceous glands (triangular arrows), etc. From the figure, it could see multiple centers of cartilage generation, and it is speculated that such multi-starting point regeneration greatly accelerates regeneration speed.

The above experimental results showed that different doses of Narciclasine could significantly promote the healing of 2 mm ear holes. The closed ear holes could regenerate tissues and tissue derivatives such as hair follicles, glands, cartilage and muscles, which confirmed the regeneration-promoting effect of Narciclasine.

### (3) Composition of NRB Promoted Regeneration of 4 mm Mouse Ear Hole at Excision

Experimental method: The method for constructing the mouse model was the same as in Example 2. Mice with 4 mm ear hole trauma were intraperitoneally injected with DMSO/NRB (NRB: Narciclasine 3 mg/kg, ATRA 20 mg/kg, BMP signaling agonist sb4 10-20 mg/kg) once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The traumatic auricles of drug-treated mice were further identified. The experimental results were shown in FIGS. 5H to 5J.

Among them, FIG. 5H showed the healing effect of the 4 mm mouse ear holes after 30 days of NRB treatment, which showed that NRB had a healing-promoting effect. After 30 days of drug treatment, the wound of the mouse ear hole was completely closed which was identified as a regeneration event. FIGS. 5I and 5J showed that the HE section staining data well indicated the regeneration structures of structures such as cartilage (long black arrows), hair follicles (asterisks), glands/sebaceous glands (triangular arrows), and muscles (dashed line boxed areas).

The above experimental results showed that the NRB small molecule composition could also promote the closure of 4 mm ear holes and promote regeneration.

### Example 6: Pancratistatin Activates ISG to Promote Regeneration of Mouse Ear Hole at Excision

### (1) Pancratistatin Activates ISG

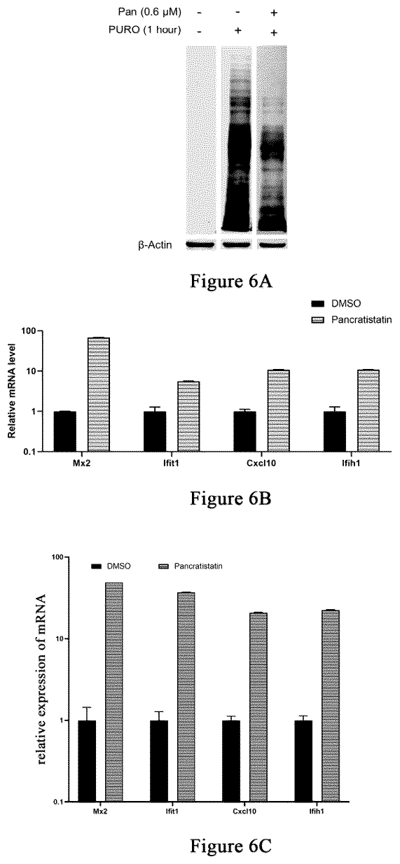

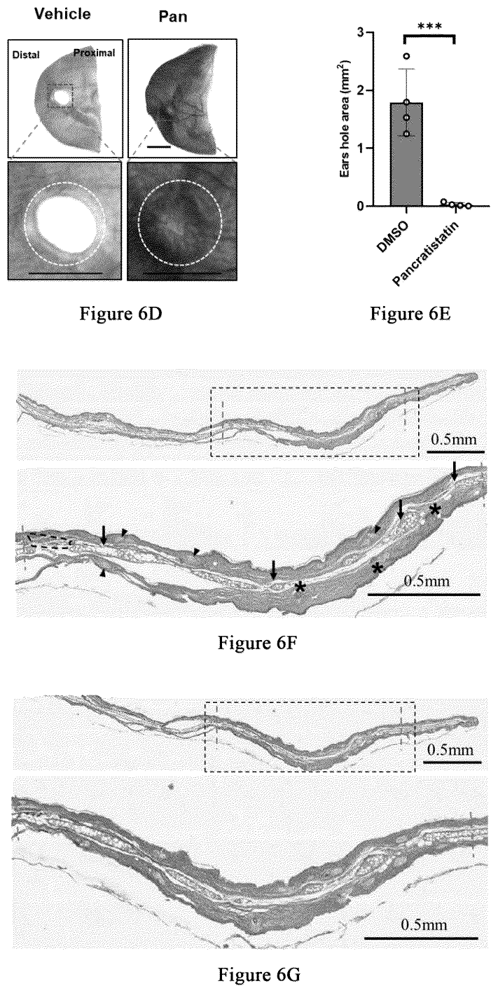

Pancratistatin, like narciclasine, is also an alkaloid of Amaryllidaceae, it could be verified to significantly inhibit overall protein translation through Puromycin (PURO) incorporation experiments first, as shown in FIG. 6A. Furthermore, quantitative qPCR experimental results showed that pancratistatin could also significantly promote the ISG gene expression, as shown in FIGS. 6B and 6C, wherein FIG. 6B showed the ISG gene expression of fibroblasts, and FIG. 6C showed the ISG gene expression of macrophages.

### (2) Pancratistatin Promotes Regeneration of 2 mm Mouse Ear Holes

Experimental method: 2 mg/kg pancratistatin (dissolved in DMSO, administration system: 2-5% DMSO+30-40% PEG400+2-5% Tween 80+saline) was intraperitoneally injected into mice with 2 mm ear hole trauma. DMSO without drug served as the control group (labeled as Vehicle). Identification of traumatic auricles of drug-treated mice was performed. The specific experimental results were shown in FIGS. 6D to 6G.

FIG. 6D showed the effect of pancratistatin administration on promoting the healing of the ear hole in the treatment group 21 days after the ear hole trauma, indicating that the ear hole was completely closed. FIG. 6E showed the closure of the ear holes observed after treatment with pancratistatin for 21-28 days. The results of FIGS. 6F (HE staining) and 6G (Masson staining) showed that the mouse ear hole was completely closed and identified as a regeneration event. The staining data well displayed the regeneration structures of multiple cartilage occurrence centers (long black arrows), hair follicles (asterisks), glands/sebaceous glands (triangular arrows), muscles (dashed line boxed areas), and other structures.

The above results show that pancratistatin small molecule could promote the closure of the ear holes and promote regeneration.

### Example 7: Interferon Activates ISG to Promote Ear Hole Regeneration

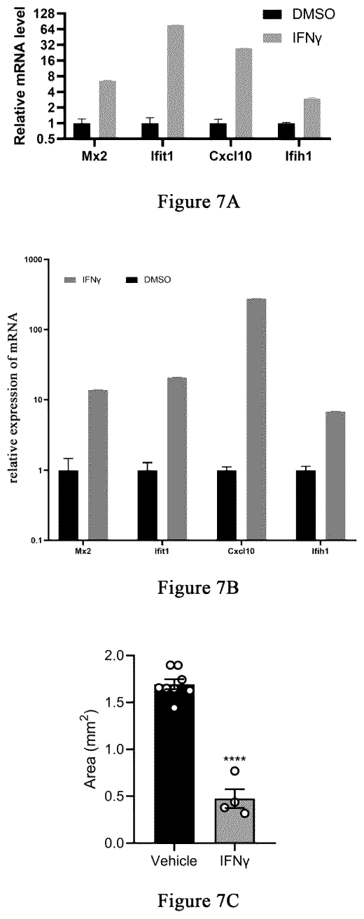

ISG is an interferon-stimulated gene. In order to verify the regeneration-promoting effect of interferon, we selected interferon (IFN) γ as a representative. First, we verified by quantitative qPCR experiments in a cell model that interferon (IFN) γ (10 ng/ml) could significantly promote the ISG gene expression. The results were shown in FIGS. 7A and 7B, wherein FIG. 7A showed the ISG gene expression of fibroblasts and FIG. 7B showed the ISG gene expression of macrophages. The regeneration-promoting effect was further verified using a mouse ear hole model by intraperitoneal injection of IFNγ (50 μg/kg) once every two days. The regeneration of the ear holes was detected on the 35th day after damage. The results were shown in FIG. 7C, indicating that interferon-γ significantly promoted the healing of ear holes after damage.

Experimental results showed that cytokine interferon activated ISG to promote ear hole regeneration.

### Example 8: S100A8/A9 Activates ISG to Promote Ear Hole Regeneration

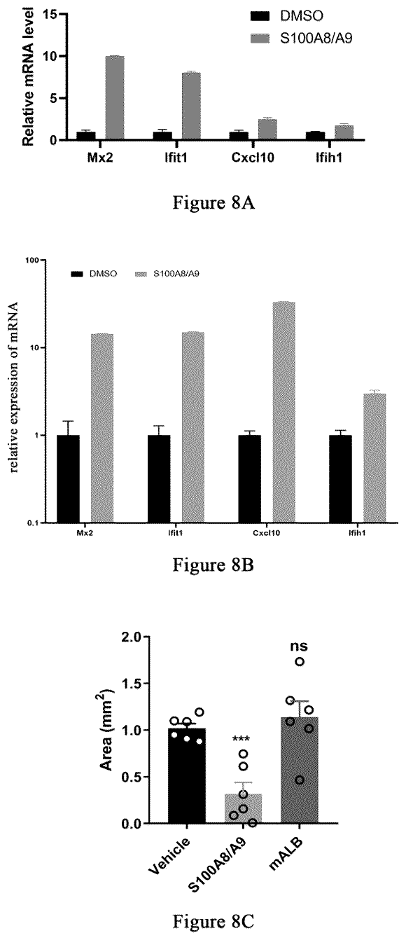

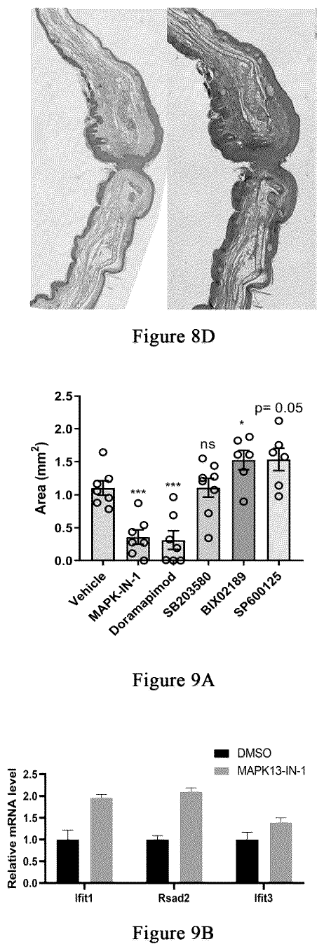

Alarmin is an induced molecule whose expression is enhanced after the body is damaged. To verify the regeneration-promoting effect of alarmin, we first verified by quantitative qPCR experiments in a cell model that S100A8/A9 (5 ng/ml) could significantly promote the ISG gene expression. The results were shown in FIGS. 8A and 8B, wherein FIG. 8A showed the ISG gene expression of fibroblasts and FIG. 8B showed the ISG gene expression of macrophages. The regeneration-promoting effect was further verified by the mouse ear hole model. PBS (Vehicle group), S100A8/A9 heterodimer (Biolegend, 765502), and mouse albumin (mALB, control) were intraperitoneally injected every two days, with injection doses of 25 and 12.5 μg/kg, respectively. The regeneration effect of the ear holes was detected on the 35th day after damage. The results were shown in FIG. 8C. On the 35th day after damage, S100A8/A9 (12.5 μg/kg) significantly promoted the healing of the ear holes, and one of the ear holes was completely closed. HE staining and Masson staining were further performed to identify the regeneration of various tissue structures, such as epidermis, dermis, hair follicles, glands, cartilage, etc., as shown in FIG. 8D.

Experimental results showed that cytokine S100A8/A9 activated ISG and promoted ear hole regeneration.

### Example 9: Inhibiting MAPK13 (p38) to Activate ISG and Promote Ear Hole Regeneration

The stress-related kinases P38, ERK5, and JNK were interfered with respectively to verify whether they have the effect of promoting ear hole regeneration. The experimental method is: the following reagents were administered to the ear hole trauma mouse model, and the healing of the ear hole was detected 21 days after the trauma.

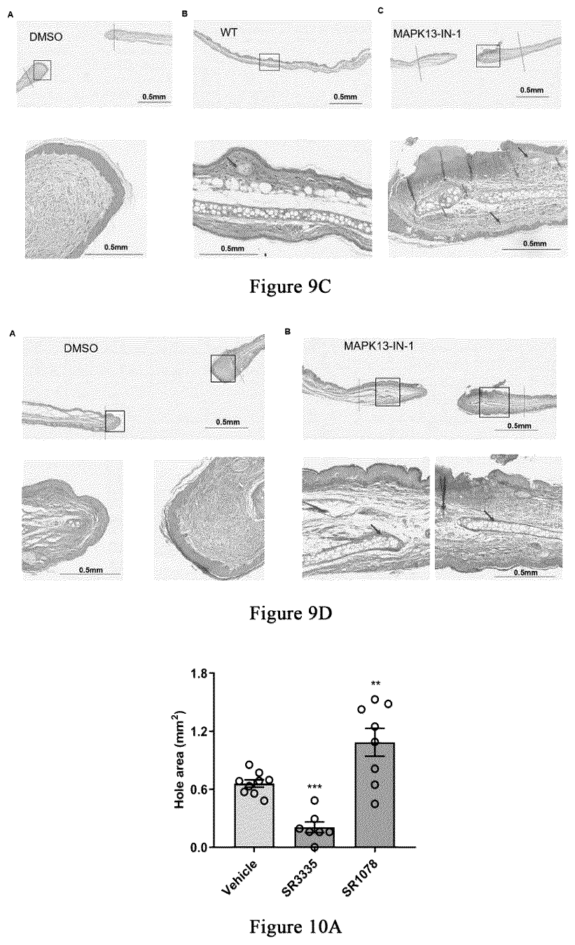

MAPK13-IN-1 (MCE, HY-12839, 5 mg/kg), a selective P38δ inhibitor; Doramapimod (MCE, HY-10320, 5 mg/kg), a P38 inhibitor; SB203580 (MCE, HY-10256, 5 mg/kg), a selective P38α/β inhibitor; BIX02189 (MCE, HY-12839, 5 mg/kg), a selective MEK5, ERK5 inhibitor; SP600125 (MCE, HY-12041, 5 mg/kg), a selective JNK inhibitor. n≥5, *p<0.05, ***p<0.001, ns: no significant difference, t-test. The experimental results were shown in FIG. 9A. Quantitative qPCR experiments further verified that the P38δ inhibitor MAPK13-IN-1 could promote the ISG gene expression (FIG. 9B). Further, HE staining images showed that after the mice with ear hole trauma were treated with MAPK13-IN-1 (20 mg/kg), various tissue structures such as the epidermis, dermis, cartilage and other connective tissue at the injured part of the mice's ear hole grew forward and connected together, and the trauma was almost completely healed (FIG. 9C). Similarly, Masson staining clearly showed the healing status after 21 days of MAPK13-IN-1 drug treatment. After treatment with MAPK13-IN-1, it could be seen that the overall arrangement of collagen fibers in the experimental group after MAPK13-IN-1 treatment is more regular and neat, closer to the wild type, arranged in bundles, and the most significant difference occurs in the regeneration of muscle fibers. Obvious regeneration of muscle fiber bundles was observed in the experimental group (dashed line boxed areas), accompanied by cartilage (arrows), and blood vessels were observed surrounding them, but no similar phenomenon was found in the control group. In general, the tissue type of the regenerated area was consistent with that of the wild type, and these indicators well indicated the occurrence of tissue regeneration events (FIG. 9D).

The experimental results showed that inhibiting P38 and P38δ to activate ISG could promote ear hole regeneration.

### Example 10: Inhibiting Retinoic Acid Receptor-Related Orphan Receptor Activation of ISG to Promote Ear Hole Regeneration

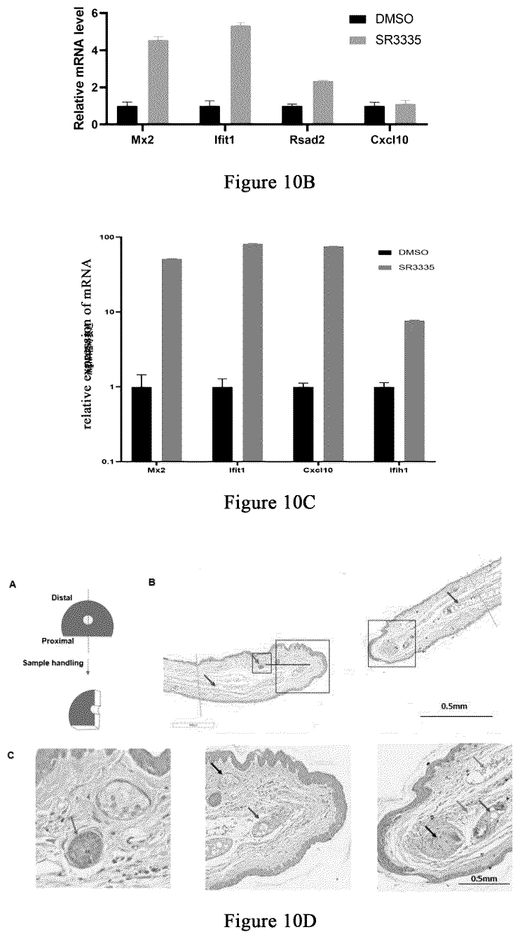

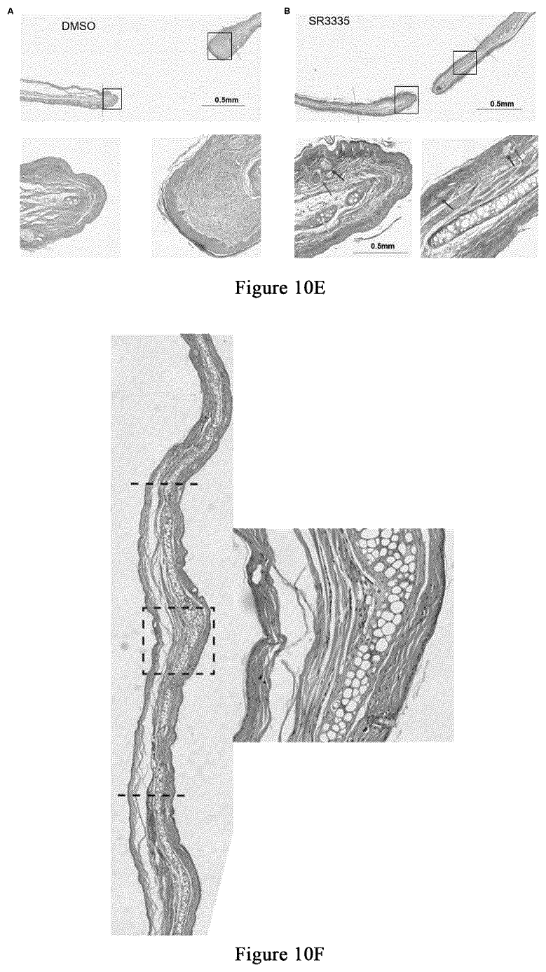

The role of retinoic acid receptor-related orphan receptor α (RORα) in ear hole regeneration was verified by activating and reversely activating the retinoic acid receptor-related orphan receptor α (RORα). The experimental method was as follows: the following reagents were administered to the ear hole trauma mouse model, and the healing of the ear holes was detected 21 days after the trauma. The results showed that reverse activation of RORα promoted the ear holes, while activation of RORα significantly inhibited regeneration, as shown in FIG. 10A. Quantitative qPCR experiments further verified that the Rora inverse agonist SR3335 could promote the ISG gene expression (FIG. 10B showed the ISG gene expression of fibroblasts and FIG. 10C showed the ISG gene expression of macrophages). Further, HE staining results showed that after treatment with SR3335, the skin, connective tissue and other tissue structures in the ear hole of the mouse with ear hole trauma grew and connected together, and the wound was almost completely healed. The regeneration of the epidermis and dermis, as well as the appearance of hair follicle structure and sebaceous glands could be observed (FIG. 10D). Masson staining clearly showed the healing status after 21 days of SR3335 drug treatment. The Masson section staining data also well characterized the regeneration structures of cartilage, hair follicles, sebaceous glands (arrows), and muscles (dashed line boxed areas) (FIG. 10E). SR3335 (MCE, HY-14413, 10 mg/kg), a selective RORα inverse agonist; SR1078 (MCE, HY-10320, 1 mg/kg), a RORα agonist; n≥7, **p<0.01, ***p<0.001, ns: no significant difference, t-test.

Rora knockout mice (introduced from Shanghai Research Center of the Southem model organisms) were used to further verify that homozygous knockout of Rora could significantly promote the closure of mouse ear holes and promote the regeneration of various tissues (FIG. 10F).

Experimental results showed that inhibiting Rora from activating ISG could promote ear hole regeneration.

### Example 11: The combination of CRB (C: Protein Synthesis Inhibitor CHX; R: RARs Activator All-Trans Retinoic Acid; B: BMP Activator BMP (Signaling Agonist sb4)) Promotes the Closure of 4 mm of Mouse Ear Holes and Regeneration at Excisions

Taking 7-week-old mice as an example, the mice were anesthetized with 5% chloral hydrate, with 10 mL/kg injected intraperitoneally according to body weight. The anesthetized mice were tied up and the ears and instruments were disinfected with 75% ethanol. The left and right auricles of the mouse were punched at the center of the auricle using a 4 mm diameter ear puncher. For mice after trauma, DMSO/CRB (The dosage of CRB is 20 mg/kg CHX, 20 mg/kg total ATRA, 10-20 mg/kg BMP signaling agonist sb4) was intraperitoneally injected once every 2 days, and the mice were anesthetized every 7 days. The proximal-distal diameter (DPD) and anterior-posterior diameter (DAP) of the mouse ear holes were measured using a vernier caliper, and the area of the mouse ear holes at excision was calculated. The area calculation formula was S=π×DPD×DAP/4. The traumatic auricles of drug-treated mice were further identified.

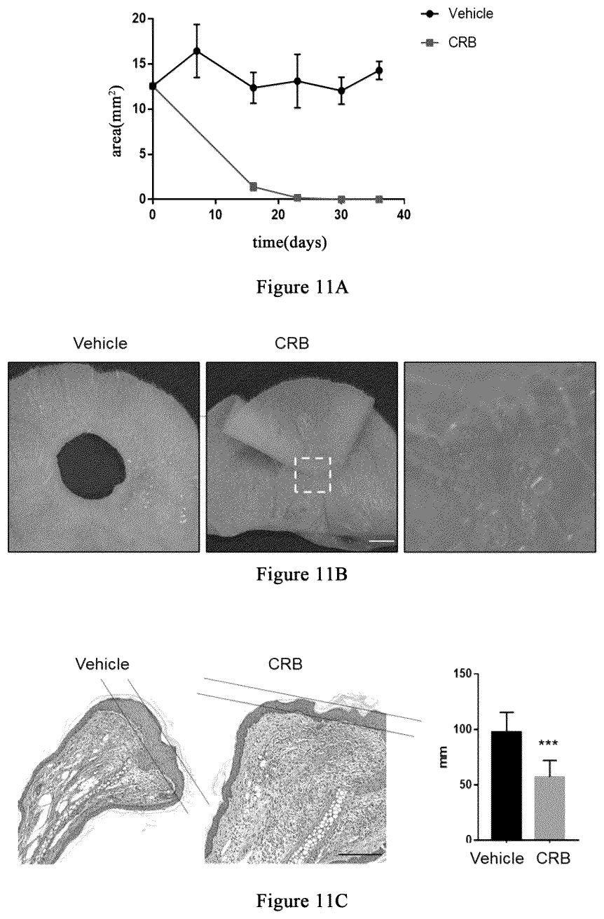

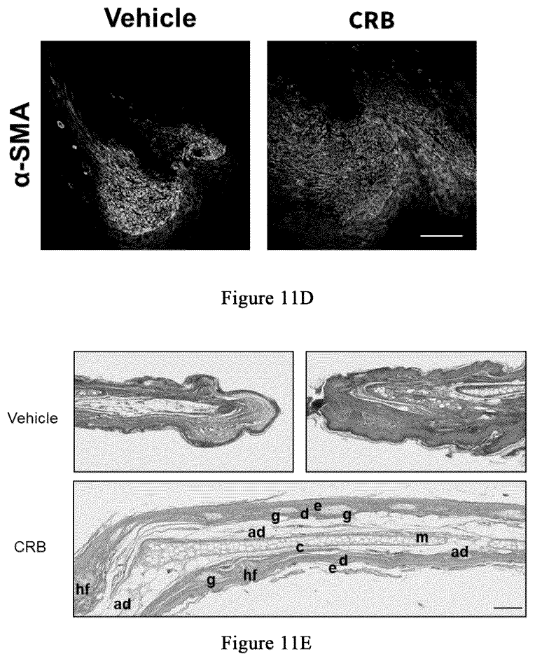

FIG. 11A showed the closure of the mouse ear holes after treatment with DMSO/CRB respectively. The results showed that CRB promoted the closure of a 4 mm diameter ear holes. After 30 days of treatment, the mouse ear hole wound were completely closed.

FIG. 11B showed the closure of the 4 mm of mouse ear holes after 30 days of drug treatment. It could be seen that the ear hole trauma of the mice treated with drugs had closed.

FIG. 11C showed the HE staining results and epidermal thickness statistics of mouse auricle tissue after 7 days of drug treatment. The scale bar was 200 μm. After drug treatment, blastema was formed in the auricle of mice and the thickness of the epidermis was significantly reduced. n≥3, t-test.

FIG. 11D showed the results of a-SMA immunofluorescence staining in mouse auricle tissue after 7 days of drug treatment. The scale bar was 100 μm. Seven days after trauma, both the control group and the drug-treated group showed a large amount of a-SMA expression. Compared with the control group, the a-SMA expression in the drug-treated group was linearly arranged in an orderly manner, while the control group was disorderly accumulated. The expression pattern was similar to the scar formation manner.

FIG. 11E showed the regeneration of various tissues after drug (CRB) induction for more than 90 days, showing the regeneration of epidermis, dermis, glands, hair follicles, muscle, cartilage, fat, and muscle. “e” indicated epidermis regeneration; “d” indicated regenerated dermis; “g” indicated regenerated gland; “ad” indicated regenerated adipose tissue; “hf” indicated regenerated hair follicle; “c” indicated regenerated cartilage; and “m” indicated regenerated muscle.

FIG. 11F further identified angiogenesis by immunofluorescence staining of the vascular marker CD31, wherein triangles indicated regenerated blood vessels.

### Example 12: Optimized Combination of Cycloheximide CHX (C) and All-Trans Retinoic Acid (R) Promotes Regeneration and Repair of Mouse Limb Outgrowth Structure

Based on the regeneration effect of ear hole, we next conducted regeneration induction experiments with more complex damage phenotypes, using limb resection as the damage model. ICR mice were subjected to limb modeling, and appropriate doses of CHX (100 mg/kg) and RAR (retinoic acid receptor) activator, all-trans retinoic acid (ATRA, 20 mg/kg) were combined and intraperitoneally administered every other day for 8 weeks to observe the regenerative phenotype.

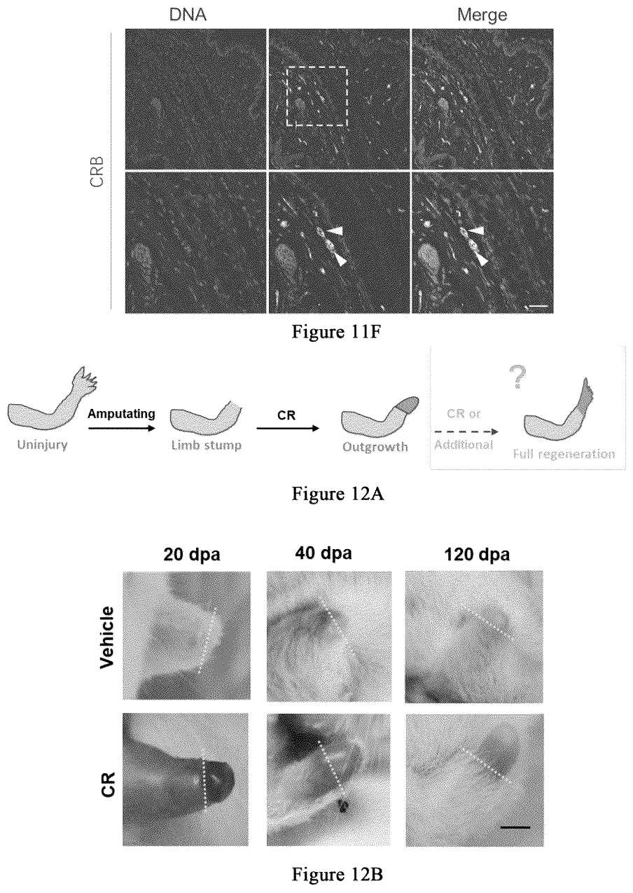

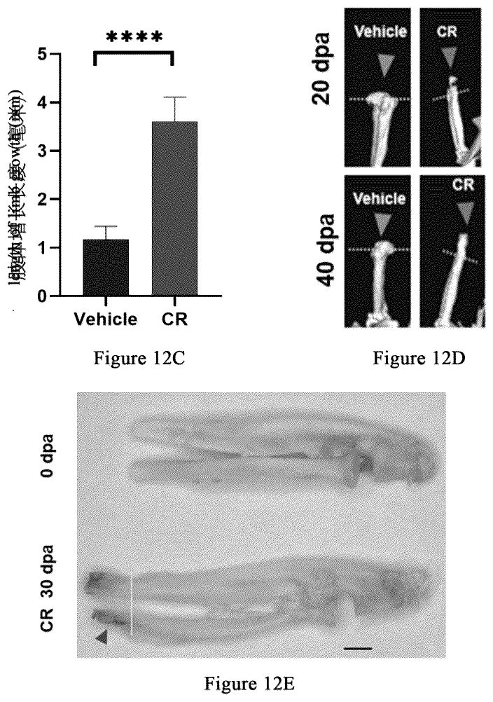

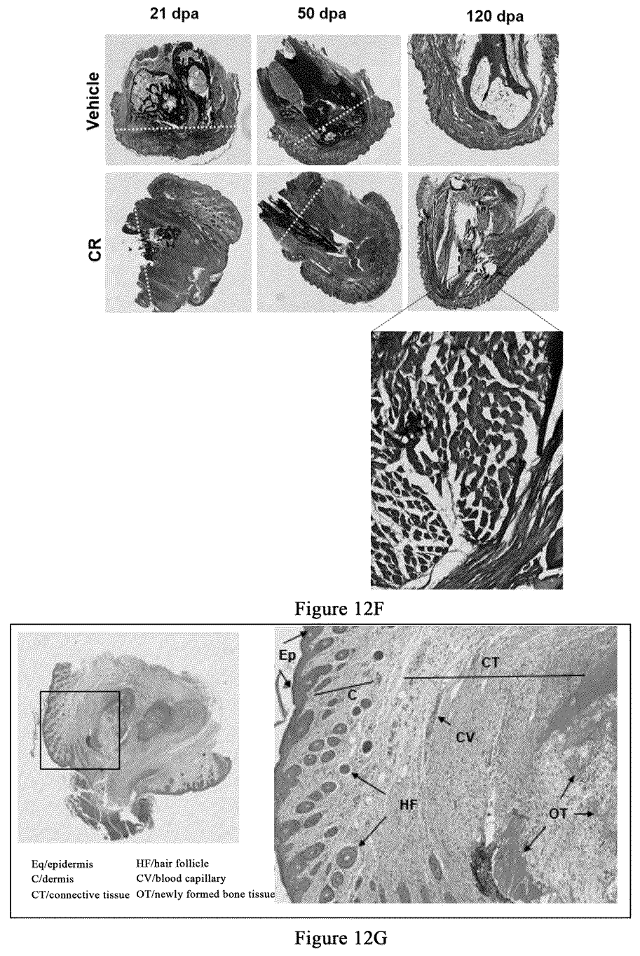

Experimental method: 8-week-old ICR mice were used as the model. Mice were anesthetized with 5% chloral hydrate, with 10 mL/kg injected intraperitoneally according to body weight. The anesthetized mouse was tied up and the left upper limb of the mouse and surgical instruments were disinfected with 75% ethanol. In order to accurately define the occurrence of regeneration, the distance from the elbow joint to the radius and ulna is measured, and 10 mm is retained. The rest to the palm, fingers and other segments are resected for modeling (the ulna and radius were two bones of the forearm. The easiest way to distinguish them is that the thumb side is the radius and the little finger side is the ulna). CHX (100 mg/kg, dissolved in DMSO or saline) and ATRA (20 mg/kg) were intraperitoneally injected every other day for 8 weeks. DMSO served as the control group, and the phenotype was continuously observed. After different days, the limb traumas of drug-treated mice were identified. The specific experimental results were shown in FIGS. 12A-G. FIG. 12A was a schematic diagram of limb modeling. FIG. 12B showed the phenomenon of limb regeneration induced by drug treatment 20, 40, and 120 days later. It could be seen that outgrowth occurred at the amputation site of the mice treated with the drug. Scale bar was 2 mm. FIG. 12C was the statistical result of the limb outgrowth length. It could be seen that the CR-treated group induced limb regeneration very well, and the length was significantly different from that of the control group. n≥3, ***p<0.001, t-test. FIG. 12D showed high-definition images of the bone tissue reconstruction process obtained by electronic computed tomography (CT) after 20 and 40 days of drug CR treatment. The CR-treated group showed a good induced growth process of limb bone tissue, and elongated reconstructed fragments of bone tissue could be observed 20 days after CR treatment (right figure, indicated by triangle arrows), while the control group showed a blunt state, showing hyperplasia and accumulation of bone tissue, and did not show a forward growth trend (left figure, indicated by triangle arrows), which was a similar occurrence to scar repair. FIG. 12E was a more direct and critical evidence of bone regeneration. After 30 days of drug CR treatment, the complete radius and ulna were taken out for observation. The results were consistent with the CT scan results, and the regeneration trend of bone tissue could be clearly seen. In addition, HE section staining revealed that the combination could promote the regeneration of multiple tissue types in the amputated limb, such as skin, hair follicles, capillaries, new bone and other tissues (FIG. 12F, “Ep” indicated epidermal regeneration; “CT” indicated connective tissue regeneration; “CV” indicated capillary regeneration; “HF” indicated hair follicle regeneration; “OT” indicated bone tissue regeneration.). FIG. 12G showed the results of Masson staining of mouse limb regeneration tissue after 21, 50, and 120 days of drug treatment. The blunt morphology of bone tissue was observed in the control group, which was consistent with the CT results. The bone tissue in the drug-treated group showed a growth trend. Importantly, regeneration of muscle tissue was observed in the experimental group at 120 days, while the control group showed disordered accumulation, and the expression pattern was similar to scar formation. Scale bar was 2 mm, n≥3, t-test.

The above results showed that cycloheximide (CHX) and all-trans retinoic acid (ATRA) promote the regeneration and repair of the outgrowth structure after limb resection, including the regeneration of bones, muscles, skin, hair follicles, capillaries, connective tissue, new bone tissue, etc.

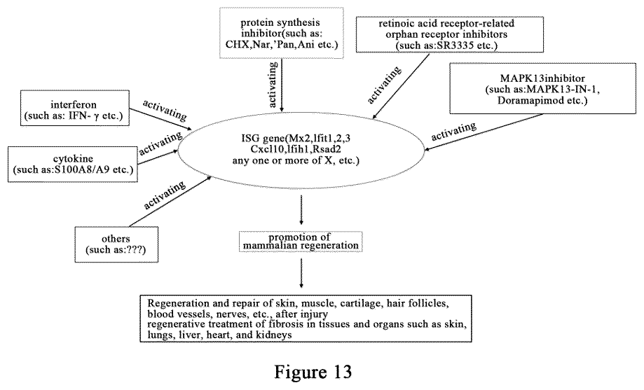

The results of the above Examples showed that various strategies for activating the ISG gene expression could promote mammalian regeneration and enhance regeneration and repair after damage. FIG. 13 was a schematic diagram showing activation of the ISG gene expression to promote regeneration capacity in mammals.

The foregoing merely illustrates the principles of the present invention, and it should be understood that the scope of the present invention is not intended to be limited to the exemplary aspects described herein, but rather to encompass all currently known and future developed equivalents.

In addition, it should be pointed out that several improvements and modifications may be made without departing from the technical principles of the present invention, and these improvements and modifications should also be regarded as within the scope of the present invention.

1-21. (canceled)

22. Use of a substance capable of up-regulating ISG gene expression in the promotion of regeneration and repair capacity of a tissue, complex structure or organ of a mammal or in the treatment of a disease related to regeneration and repair of a tissue, complex structure or organ of a mammal.

23. The use according to claim 22, wherein the substance capable of up-regulating the ISG gene expression is any one or more selected from the group consisting of: a MAPK inhibitor, retinoic acid receptor-related orphan receptor inhibitor, protein synthesis inhibitor, interferon (IFNγ, β, λ), and alarmin (S100A8/A9) protein.

24. The use according to claim 22, wherein the promotion of regeneration and repair capacity of the tissue, complex structure or organ of the mammal is achieved by inducing activation of the TBK1-IRF3 pathway, preferably by inhibiting protein synthesis.

25. The use according to claim 22, wherein the promotion of regeneration and repair capacity of the tissue, complex structure or organ of the mammal is achieved by inhibiting the p38 MAPK pathway, preferably by inhibiting P38δ.

26. The use according to claim 22, wherein the promotion of regeneration and repair capacity of the tissue, complex structure or organ of the mammal is achieved by reverse activation of retinoic acid receptor-related orphan receptor α (RORα).

27. The use according to claim 22, wherein the regeneration and repair is promotion of the regeneration of a tissue, complex structure or organ after tissue or organ resection or damage.

28. The use according to claim 22, wherein
the tissue is skin, fat, muscle, bone, hair follicle, blood vessel or nerve;
the complex structure is at least two or more selected from the group consisting of: body structure comprising skin, hair follicle, gland, cartilage, muscle, fat, blood vessel, nerve, and limb; and
the organ is lung, liver, heart, pancreatic islet or kidney.

29. The use according to claim 22, wherein the complex structure is an ear, a limb, a finger, an eye, or a nose.

30. The use according to claim 22, wherein the regeneration and repair is promotion of regeneration and repair after skin damage, hair regeneration after hair loss, regeneration and repair of cartilage and muscle damage, regeneration of fibrosis of lung, liver, skin, heart, kidney and muscle, and regeneration after blood vessel, nerve and limb damage.

31. The use according to claim 22, wherein the disease is skin scald, skin trauma, skin burn, hair loss, cartilage and muscle damage, liver fibrosis, pulmonary fibrosis, or limb damage.

32. The use according to claim 23, wherein the MAPK inhibitor is any one or more selected from the group consisting of: a P38 inhibitor and a selective P38δ inhibitor, preferably the P38 inhibitor is Doramapimod, and the selective P388 inhibitor is MAPK13-IN-1.

33. The use according to claim 23, wherein the retinoic acid receptor-related orphan receptor inhibitor is a selective RORα inverse agonist, preferably SR3335.

34. The use according to claim 23, wherein the protein synthesis inhibitor is any one or more selected from the group consisting of: cycloheximide (CHX), anisomycin (Ani), didemnin B (DIDB), bouvardin (BVD), narciclasine, and pancratistatin.

35. A composition, wherein it comprises a protein synthesis inhibitor, and an all-trans retinoic acid and a BMP activator, preferably the protein synthesis inhibitor is cycloheximide (CHX), and the BMP activator is BMP signaling agonist sb4.

36. The composition according to claim 35, wherein based on 1 part by weight of the cycloheximide in the composition, the amount of the all-trans retinoic acid is 0.25 to 8 parts by weight, and the amount of the BMP signaling agonist sb4 is 0.25 to 4 parts by weight.

37. The composition according to claim 35, wherein the composition is administered by intraperitoneal injection, intravenous injection, oral gavage, oral administration or skin application.

38. Use of the composition of claim 35 in the promotion of regeneration and repair capacity of a tissue, complex structure or organ of a mammal, or in the preparation of a medicament or reagent for promoting regeneration and repair capacity of a tissue, complex structure or organ of a mammal, or in the preparation a medicament or reagent for treating a disease related to regeneration and repair of a tissue, complex structure or organ of a mammal.

39. A method for promoting regeneration and repair of a tissue, complex structure or organ of a mammal, comprising administering a substance capable of up-regulating ISG gene expression or a composition comprising the substance capable of up-regulating the ISG gene expression to a subject in need thereof.

40. The method according to claim 39, wherein the substance capable of up-regulating the ISG gene expression is any one or more selected from the group consisting of: a MAPK inhibitor, retinoic acid receptor-related orphan receptor inhibitor, protein synthesis inhibitor, interferon (IFNγ, β, λ), and alarmin (S100A8/A9) protein.

41. The method according to claim 39, wherein the composition comprising the substance capable of up-regulating the ISG gene expression is a composition comprising a protein synthesis inhibitor, and an all-trans retinoic acid and a BMP activator, preferably the protein synthesis inhibitor is cycloheximide (CHX), and the BMP activator is BMP signaling agonist sb4.
